(19) United States
(12) Plant Patent Application Publication
(10) Pub. No.: US 2018/0160591 P1
(43) Pub. Date: Jun. 7, 2018

(54) Strawberry plant named 'FLORIDA BEAUTY'

(50) ;

(71) Applicants: Florida Foundation Seed Producers, Inc., Marianna, FL (US); State of Queensland acting through The Department of Agriculture and Fisheries, Brisbane QLD (AU)

(72) Inventors: Vance M. Whitaker, Brandon, FL (US); Mark Herrington, Nambour (AU)

(21) Appl. No.: 15/530,158

(22) Filed: Dec. 7, 2016

(51) Int. Cl. A01H 5/08

(52) U.S. Cl. PLT/208

(57) ABSTRACT

A new and distinct variety of strawberry (Fragaria×ananassa), which originated from seed produced by a hand-pollinated cross between AU 2010-119 and ‘Florida Radiance’. The new strawberry, named ‘Florida Beauty’, is distinguished by its compact growth habit; steady yield; fruit that are exceptionally and consistently well-shaped, bright medium-red, and having an exceptional, balanced flavor when grown in west central Florida or other areas that have a subtropical climate similar to that of west central Florida.

Latin name of the genus and species of the plant claimed: Fragaria×ananassa Duchesne.

Variety denomination: ‘Florida Beauty’.

### BACKGROUND OF THE INVENTION

The present invention relates to a new and distinct variety of strawberry (Fragaria ×ananassa Duchesne) named ‘Florida Beauty.’ This new strawberry plant is distinguished by its compact growth habit and ability to produce steady yields of fruit that are consistently shaped and have exceptional flavor when grown in west central Florida. Asexual propagation was performed at Balm, Fla. where the selection was made and plants were tested. Contrast is made to ‘Florida Radiance’ (U.S. Plant Pat. No. 20,363) and ‘Florida127’ (U.S. Plant Pat. No. 25,574), currently the dominant varieties in Hillsborough County, Fla., for reliable description. This new variety is a promising candidate for commercial success because it can be planted earlier than other varieties due to its compact plant habit and because it produces flavorful fruit that are evenly colored and consistently shaped during the entire Florida market window.

This strawberry plant (genotype) originated in a strawberry breeding plot in Balm, Fla. The seed parent was AU 2010-119, an unreleased breeding selection with the ability to produce fruit with excellent flavor and shape. The pollen parent was ‘Florida Radiance’ (U.S. Plant Pat. No. 20,363), a strawberry cultivar with high early yields, steady yield pattern, and low vegetative vigor. The seeds resulting from the controlled hybridization were germinated in a greenhouse, and the resulting seedlings were planted and allowed to produce daughter plants by asexual propagation (i.e. by runners). Two daughter plants from each seedling were transplanted to raised beds, where they fruited. ‘Florida Beauty’ (as represented by two daughter plants from the original seedling) exhibited compact plant habit and steady yields of consistently shaped fruit, and therefore was selected for further evaluation. ‘Florida Beauty’ was selection number 5 of the 121^{st} cross in the 2012-2013 seedling trial, and thus was given the breeding trial designation of FL 12.121-5. ‘Florida Beauty’ has been asexually propagated annually by runners, and further test plantings have established that the vegetative and fruit characteristics of the propagules are identical to the initial daughter plants.

### SUMMARY OF THE INVENTION

‘Florida Beauty’, when grown in a subtropical fall and winter climate, is set apart from all other strawberry plants by a combination of the following characteristics: compact growth habit; steady yield; and fruit that are exceptionally and consistently well-shaped, bright medium-red, and have an exceptional balanced flavor.

### BRIEF DESCRIPTION OF THE DRAWINGS

The accompanying photograph shows a typical specimen of a 5-month-old plant and fruit as seen in February, 2016, in west central Florida.

FIG. 1—Shows whole plants, including leaves, inflorescences, and fruit at varying stages of ripeness.

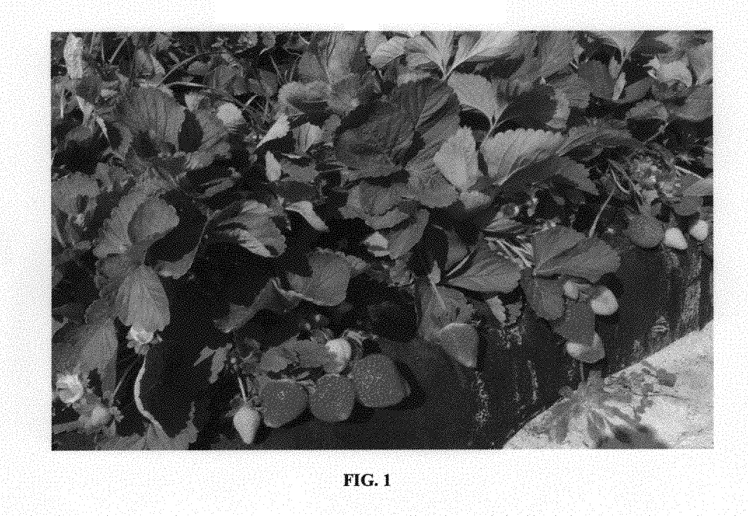

### DETAILED BOTANICAL DESCRIPTION

The following botanical description is that of plants of the claimed variety grown under the ecological conditions (warm days, cool nights) prevailing in Balm, Fla., during the winter production season. Colors are objectively described using the L*a*b* color scale with a colorimeter.
Phenotypic description of Fragaria×ananassa Duchesne (‘Florida Beauty’):
Plant:
   Average height.—15 cm.
   Average width.—25 cm.
   Growth habit.—Compact, semi-spreading plant that is not overly dense.
   Number of crowns/plant.—4 to 7 depending on seasonal conditions.
   Vigor.—Low to Medium.
Leaf:
   Overall description.—Pinnately compound with three leaflets.
Petiole:
   Average length.—15.5 cm.
   Average diameter.—2.5 mm.
   Pubescence.—Light to medium.
   Pubescence density.—Moderate to sparse.
   Pose of hairs.—Perpendicular.
   Texture.—Smooth.
   Anthocyanin.—Minimal to absent.
   Color.—Medium-light green (L*=58.9, a*=−13.3, b*==32.1).
Petiolule:
   Length.—Terminal leaflet 8 mm; lateral leaflets, 5 mm.
   Diameter.—2 mm.
Stipule:
   Length.—33 mm.
   Width.—18 mm along base of petiole attachment.
   Anthocyanin.—Slight to absent.
Terminal leaflet:
   Average length.—82 mm.
   Average breadth.—69 mm.
   Length/width ratio.—1.19.
   Shape in cross section.—Concave.
   Color, upper surface.—Medium-light green (L*=33.2, a*=−11.6, b*=14.5).
   Glossiness.—Slight gloss.
   Base shape.—Cuneate to slightly rounded.
   Apex descriptor.—Rounded.
   Pubescence density.—Sparse.
   Texture.—Moderately smooth.
   Venation pattern.—Pinnate.
Secondary leaflets:
   Average length.—78 mm.
   Average breadth.—80 mm.
   Length/width ratio.—0.96.
   Shape in cross section.—Concave.
   Color, upper surface.—Medium-light green (L*=33.7, a*=−11.7, b* =14.8).
   Glossiness.—Slight gloss.
   Base shape.—Oblique rounded.
   Apex descriptor.—Obtuse.
   Pubescence density.—Sparse.
   Texture.—Moderately smooth.
   Venation pattern.—Pinnate.
Leaflet margins: Crenate, with an average of 22 serrations per terminal leaflet and 22 per secondary leaflet.
Stolons:
   Number of daughter plants.—20-30 depending on environmental conditions.
   Anthocyanin.—Variable; light to moderate.
   Thickness.—2-3 mm.
   Pubescence.—Light to moderate.
Inflorescence:
   Time of flowering.—Partial to full remontancy.
   Position relative to canopy.—Flowers open at or below canopy height.
   Branching of the inflorescence.—At or very close to the crown.
Petals:
   Number.—5 to 6.
   Length.—12 mm.
   Width.—12 mm.
   Mean diameter of the corolla (i.e. the petals collectively).—23.3 mm.
   Number of stamens.—Average of 23.
Calyx:
   Diameter of calyx relative to corolla.—60%.
   Color.—Medium green (L*=44.8, a*=−16.1, b*=26.5).
Pedicels:
   Attached to mature primary fruit.—9.5 to 13 cm long depending on time of season. At peak production, the plant will have several crowns, each producing a truss, and each truss will have 3-7 pedicels. Inforescences branch very close to the crown, rendering the peduncle rarely visible.
Fruit:
   Mean fruit weight.—Slightly less than ‘Florida Radiance’ (Table 1).
   Shape.—Medium conical to cordate in shape.
   Weight, primary fruit.—27-37 g.
   Weight, secondary and tertiary fruit.—10-23 g.
   Fruit flavor.—Usually sweeter than ‘Florida Radiance’ and having strawberry flavor never lower than ‘Florida Radiance’ and usually not different from ‘Florida127’ which is the current commercial flavor standard in Florida (Table 2).
   Fruit cavity.—Rare.
   Achenes.—Slightly sunken, giving the fruit a smooth appearance.
   External fruit color.—Glossy red (a*=39A).
   Internal fruit color.—Medium red (a*=17.4).
   Evenness of color.—Consistently even.
   Flesh and skin firmness at full ripe stage.—Moderately firm.
   Rain damage.—More resistant to cracking of the fruit by rain than ‘Florida127’ and similar to ‘Florida Radiance’.
Total yield: Not different from ‘Florida127’ in 2015-16 (a season with above-average temperatures, to which ‘Florida Beauty’ is best adapted at typical planting dates) but lower than ‘Florida127’ and ‘Florida Radiance’ in 2014-15 (a cooler season) (Table 1).
Preferred planting period: September 25^{th} to October 5^{th} in west-central Florida.
   Nursery performance.—‘Florida Beauty’ is expected to perform well in nursery situations due to the production of a moderate number of runners, which result in sturdy, compact daughter plants. This is in contrast to ‘Florida Radiance’, whose daughter plants have weak petioles that are susceptible to breakage during digging and handling.
Disease resistance: ‘Florida Beauty’ is moderately susceptible to Botrytis fruit rot (caused by Botrytis cinerea), similar to ‘Florida Radiance’. ‘Florida Beauty’ is moderately resistant to anthracnose fruit rot (caused by Colletotrichum acutatum).

TABLE 1
Performance of three strawberry genotypes during the 2014-15 and 2015-16 seasons in Balm, Florida

| | Marketable yield (g/plant) | | | | | | |
|---|---|---|---|---|---|---|---|
| Cultivar | November | December | January | February | March | Total | Wt/fruit(g)^{z} |

2014-15

| | | | | | | | |
|---|---|---|---|---|---|---|---|
| F. Beauty | 7.4 a | 55.5 b | 149.0 b | 180.4 b | 209.6 a | 601.3 b | 22.3 c |
| Florida127 | 0.0 b | 95.8 a | 212.7 a | 288.4 a | 206.6 a | 803.6 a | 30.8 a |
| F. Radiance | 1.6 b | 60.5 b | 152.2 b | 327.0 a | 210.9 a | 749.7 a | 25.0 b |

2015-16

| | | | | | | | |
|---|---|---|---|---|---|---|---|
| F. Beauty | 39.6 a^{y} | 87.5 a | 68.9 a | 187.7 a | 129.3 a | 490.2 a | 18.9 b |
| Florida127 | 16.7 b | 75.3 a | 38.2 b | 139.4 a | 129.4 a | 411.9 a | 21.5 a |

^{z}Mean fruit weight was determined by dividing total marketable fruit yield per plot by total marketable fruit number per plot.
^{y}Means are based on four replications of 10 plants each in 2014-15 and on five replications of 10 plants each in the other seasons. Means separation within columns is by Tukey's HSD test, P ≤ 0.05. Plants of ‘Florida Radiance’ were not available for the 2015-16 trial

TABLE 2
Trained sensory panel ratings from five dates over two harvest seasons

| | | | | | |
|---|---|---|---|---|---|
| | | | | Strawberry | Green |
| Cultivar | Firmness | Sweetness | Sourness | flavor | flavor |

January 2015

| | | | | | |
|---|---|---|---|---|---|
| F. Beauty | 5.7 a^{z} | 5.9 a | 4.5 a | 5.1 a | 1.8 a |
| Florida127 | 5.6 a | 5.2 a | 4.7 a | 4.4 a | 1.8 a |
| F. Radiance | 4.9 a | 4.9 a | 4.5 a | 4.1 a | 1.6 a |

February 2015

| | | | | | |
|---|---|---|---|---|---|
| F. Beauty | 6.6 a | 5.2 a | 4.8 a | 4.3 ab | 2.6 a |
| Florida127 | 5.5 a | 5.4 a | 4.2 a | 4.8 a | 1.9 a |
| F. Radiance | 5.6 a | 3.9 b | 5.0 a | 3.6 b | 2.4 a |

March 2015

| | | | | | |
|---|---|---|---|---|---|
| F. Beauty | 3.6 b | 4.3 a | 4.5 a | 3.9 a | 1.8 a |
| Florida127 | 4.5 b | 5.0 a | 4.5 a | 4.3 a | 1.8 a |
| F. Radiance | 5.6 a | 3.1 b | 4.7 a | 3.0 b | 2.5 a |

February 2016

| | | | | | |
|---|---|---|---|---|---|
| F. Beauty | 5.1 a | 5.4 a | 5.4 a | 4.1 a | 2.2 a |
| Florida127 | 4.7 ab | 5.2 a | 4.8 a | 4.1 a | 2.1 a |
| F. Radiance | 4.0 b | 4.8 a | 5.4 a | 4.1 a | 2.2 a |

March 2016

| | | | | | |
|---|---|---|---|---|---|
| F. Beauty | 5.3 a | 3.9 a | 6.3 a | 3.8 a | 2.6 b |
| Florida127 | 5.4 a | 4.7 a | 5.0 b | 3.6 a | 2.0 b |
| F. Radiance | 5.9 a | 2.8 b | 6.9 a | 2.0 b | 4.0 a |

^{z}Mean separations are within harvest dates and within columns by Tukey's HSD test, P ≤ 0.05.

What is claimed is:

1. A new and distinct strawberry plant as illustrated and described herein, characterized by: (1) a compact growth habit; (2) steady yield; and (3) fruit that are exceptionally and consistently well-shaped, bright red in color and having an exceptional, balanced flavor when grown in west central Florida.
